(19) United States
(12) Patent Application Publication
(10) Pub. No.: US 2004/0158131 A1
(43) Pub. Date: Aug. 12, 2004

(54) Medical recording device carried on one's person

(75) Inventor: Wen-Hsiang Hsiao, Taipei City (TW)

Correspondence Address: BACON & THOMAS, PLLC, 625 SLATERS LANE, FOURTH FLOOR, ALEXANDRIA, VA, 22314

(73) Assignee: WEM Technology Inc., Taipei (TW)

(21) Appl. No.: 10/359,148

(22) Filed: Feb. 6, 2003

(51) Int. Cl. A61B 5/00

(52) U.S. Cl. 600/300

(57) ABSTRACT

The medical recording device carried on one's person is provided for convenience of reading medical records of a patient through a reading device, and is provided with a main unit having an inserting end connecting to a connecting port of a main frame, the main unit is provided therein with a flash memory storage circuit for reading, storing medical records, the flash memory storage circuit is serially connected with a serial connective line concentrating circuit, the serial connective line concentrating circuit is connected with the inserting end provided on one end of the main unit; in this way, the medical recording device carried on one's person is completed.

### BACKGROUND OF THE INVENTION

1. Field of the Invention

The present invention is related to a medical recording device carried on one's person, and especially to such a device which is carried on one's person and is a storage device of personal medical records to assure integrity of the medical records, so that medical workers can effectively control the situation of a patient when the patient comes for curing or has to be saved in an emergency,

2. Description of the Prior Art

When people go to doctors, they need to fill in diagnosis record tables at clinics in advance; after diagnosis staffs checkout the causes of deceases, the records are filled in the record tables for reference of subsequent diagnoses thereafter. If each time a patient changes for a different clinic when he goes for a diagnosis, a new diagnosis record table shall be filled in; and the diagnosis staffs shall rewrite the diagnosis records on the new diagnosis record tables after diagnoses. They just cannot obtain the preceding diagnosis records, thereby, non-unified diagnoses may be created; and thereby the patient will be unable to get a right therapy against the cause of his decease, the efficiency of medical treatment will be reduced.

Particularly, when a patient has the cause of decease of a malign virus or more serious cause of decease, his doctor will be unable to control the preceding diagnosis situations of the patient; if it is the case that the patient seeks medical treatment at a nearby medical unit under an emergency without a preceding diagnosis record, wrong diagnosis might be induced by the diagnosis staffs and seriously affects the life of the patient. Therefore, there have been developed medical IC cards; such medical IC cards mainly are used to read the medical records of a patient stored in a remote terminal by a computer on-line operation mode. By this mode, the above stated defects resided in diagnoses can be effectively gotten rid of, however, the IC cards can only have the completed medical records of a patient obtained by such computer on-line operation mode, hence when a terminal of such computer on-line operation mode is broken of connect ion or off-line, the present terminal will be unable to get the completed medical records, this similarly can affect the time efficiency of diagnosis of the patient.

### SUMMARY OF THE INVENTION

In view of the above statements, the inventor of the present invention provides a medical recording device carried on one's person against the above stated defects based on his professional experience of years in studying, designing and manufacturing same kind of products and after hard study, developing, as well as repeated experiments and tests.

Therefore, the medical recording device carried on one's person of the present invention has a main unit with an inserting end connecting to a connecting port of a main frame; the main unit is provided therein with a flash memory storage circuit for reading, storing medical records, the flash memory storage circuit is serially connected with a serial connective line concentrating circuit, the serial connective line concentrating circuit is connected with the inserting end provided on one end of the main unit; thereby the medical recording device carried on one's person is completed.

Therefore, the primary object of the present invention is to render medical workers to effectively control the situation of a patient by using the medical recording device carried on one's person, thereby the deceases of the patient can be surely cured or given with a first aid.

The present invention will be apparent in its contents and effects to be achieved after reading the detailed description of the preferred embodiment thereof in reference to the accompanying drawings.

### BRIEF DESCRIPTION OF THE DRAWINGS

FIG. 1 is a perspective schematic view showing a medical recording device of the present invention;

FIG. 2 is a circuit diagram showing allocation in a serial connective line concentrating circuit of the present invention;

FIG. 3 is a circuit diagram showing allocation in a flash memory storage circuit of the present invention;

FIG. 4 is a flow chart showing the situation of use of the present invention;

FIGS. 5 and 6 are examples of medical recording tables for the present invention.

### DETAILED DESCRIPTION OF THE PREFERRED EMBODIMENT

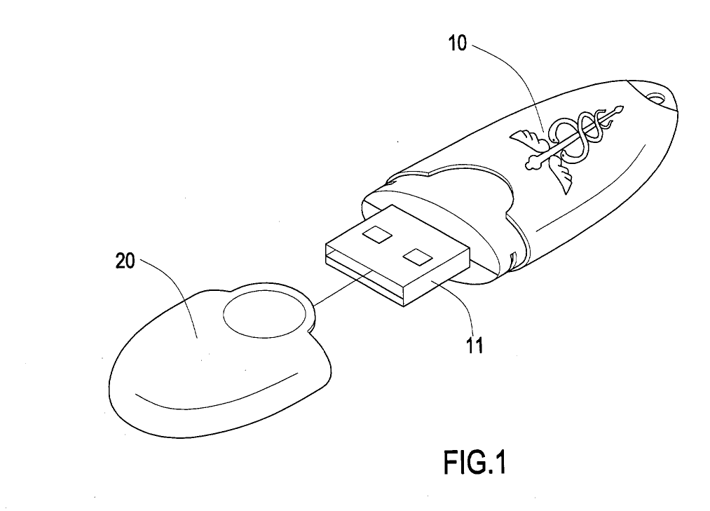

Referring firstly to FIGS. 1, 2 and 3, the medical recording device carried on one's person of the present invention has a main unit 10 with an inserting end 11 connecting to a connecting port of a main frame. The main unit 10 is provided therein with a serial connective line concentrating circuit 12 integrated with a flash memory storage circuit 13 for reading, storing medical records. The inserting end 11 is connected with the serial connective line concentrating circuit 12 in the main unit 10, and is exposed out of the main unit 10 to connect with the connecting port of the main frame. A cover 20 at one side of the inserting end 11 is connected with the main unit 10 to protect intactness of the inserting end 11.

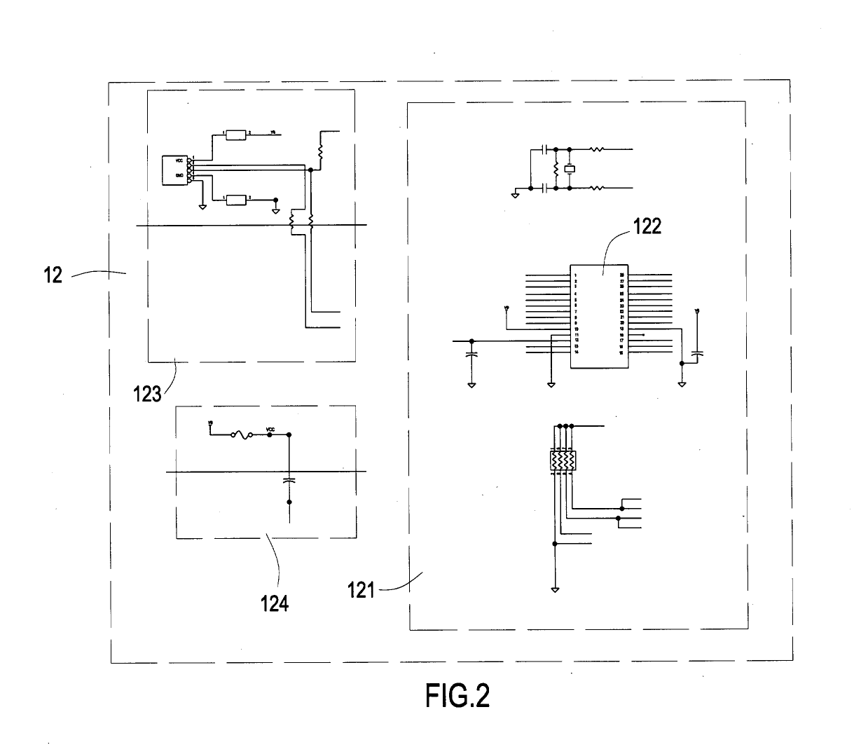

The serial connective line concentrating circuit 12 (referring to FIG. 2) is provided therein with a serial distribution control circuit 121, a serial connective interface 122, a serial connective line 123 and a power supply protecting loop 124 integrated with one another. The inserting end 11 is connected with the serial connective line 123 to make the form of a connector as shown in FIG. 1; then the entire medical recording device can be insertion connected to the connecting port of a reading device such as a computer for use.

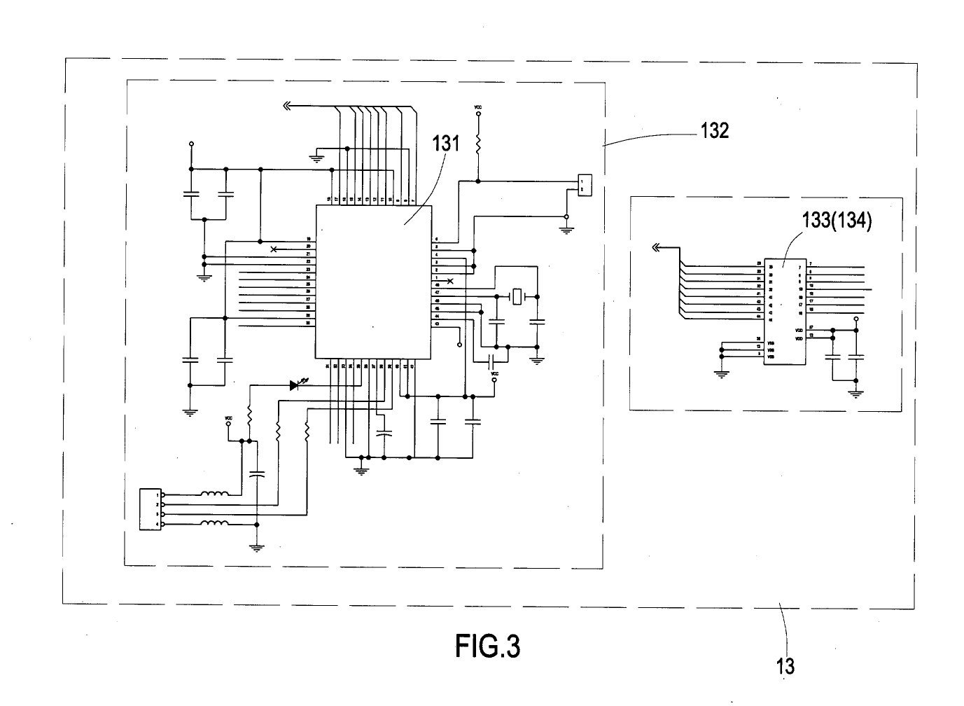

As to the flash memory storage circuit 13 (referring to FIG. 3), it is provided therein with a microprocessor 131, a flash memory control circuit 132, a memory 133 and a flash memory 134 integrated with one another (in the present invention, the memory 133 and the flash memory 134 can be formed from a same integrated circuit). Thereby, the entire recording device can be used for connecting of a serial connective interface in the flash memory storage circuit 13 to form integration of the serial connective line concentrating circuit 12 with the flash memory storage circuit 13. Thereby, the entire recording device can be insertion connected with the reading device at any time for reading the medical records stored in the flash memory storage circuit 13.

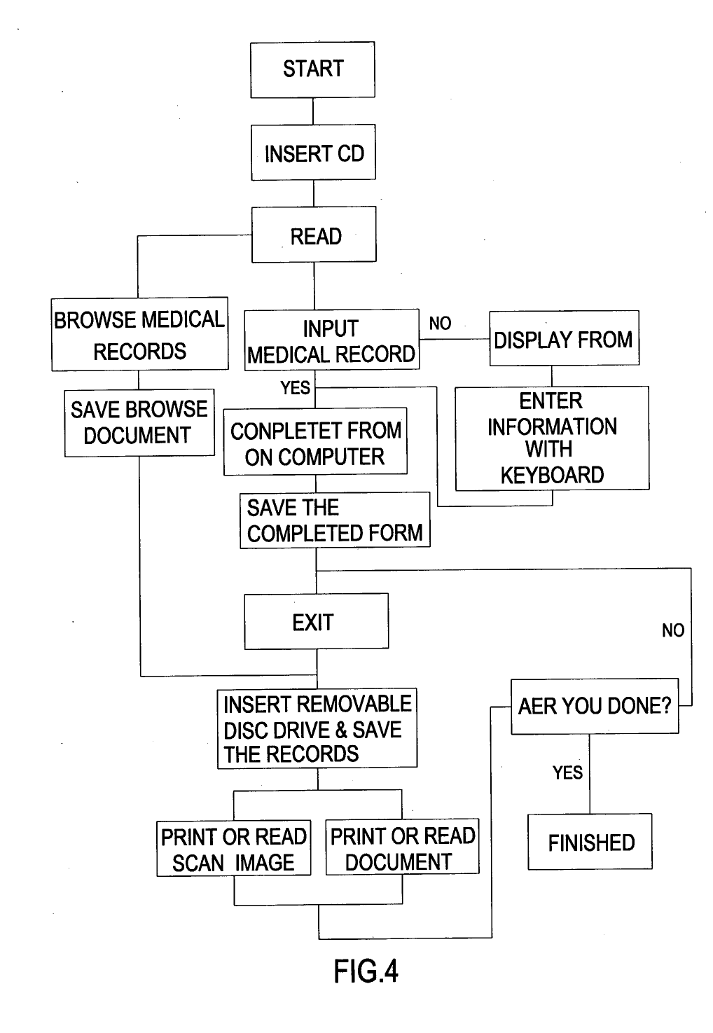

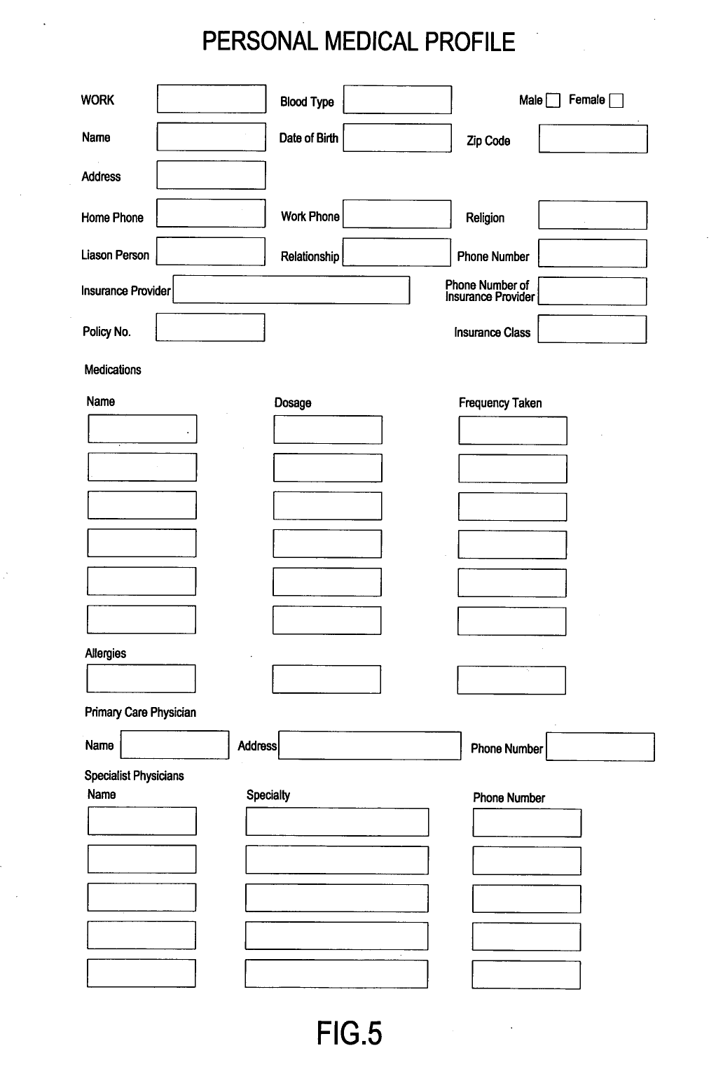

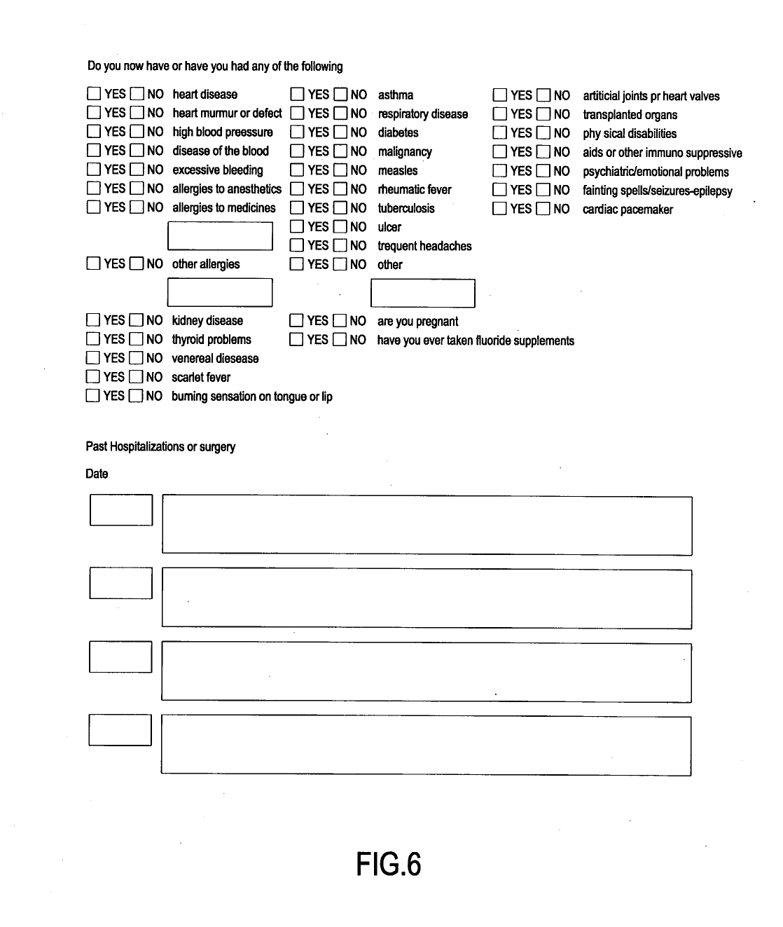

Using of the recording device is shown in FIG. 4, in the first place, the recording device is inserted into the reading device for reading the medical records stored in the flash memory storage circuit 13 in the recording device (referring to FIGS. 5 and 6). When in reading, the user has an option to browse or fill in medical records; if he selects to fill in medical records, after the medical records are filled in by inputting, then the medical records are stored in the flash memory storage circuit 13. The medical records stored in the flash memory storage circuit 13 can also be printed or be selected to be read directly in the reading device; thereby, the preceding diagnosis records of a patient can be obtained at any time when the patient comes, thereby the deceases of the patient can be cured.

The medical recording device carried on one's person of the present invention is formed by providing a main unit with an inserting end connecting to a connecting port of a main frame, and the main unit is provided therein with a flash memory storage circuit for reading, storing medical records, the flash memory storage circuit is serially connected with a serial connective line concentrating circuit, the serial connective line concentrating circuit is connected with the inserting end provided on one end of the main unit; in this way, the medical recording device carried on one's person is completed.

The names of the members composing the present invention are only for illustrating the embodiment of the present invention, and not for giving any limitation to the scope of the present invention. It will be apparent to those skilled in this art that various equivalent modifications or changes without departing from the spirit of this invention shall also fall within the protected scope of the appended claims.

1. A medical recording device carried on one's person, said device has a main unit with an inserting end connecting to a connecting port of a main frame, wherein:
said main unit is provided therein with a flash memory storage circuit for reading, storing medical records, said flash memory storage circuit is serially connected with a serial connective line concentrating circuit, said serial connective line concentrating circuit is connected with said inserting end provided on one end of said main unit; in this way, said medical recording device carried on one's person is completed.

2. The medical recording device carried on one's person as in claim 1, wherein:
said serial connective line concentrating circuit is provided therein with a serial distribution control circuit, a serial connective interface, a serial connective line, a power supply protecting loop and a plurality of connecting ports integrated with one another.

3. The medical recording device carried on one's person as in claim 1, wherein said flash memory storage circuit is provided therein with a microprocessor, a flash memory control circuit, a memory and a flash memory integrated with one another.

4. The medical recording device carried on one's person as in claim 1, wherein a cover is provided at one side of said inserting end and is connected with said main unit to protect intactness of said inserting end.
